(19) United States
(12) Patent Application Publication
(10) Pub. No.: US 2025/0143570 A1
(43) Pub. Date: May 8, 2025

(54) Ergonomic Indirect Ophthalmoscope

(71) Applicants: United States Government As Represented By The Department Of Veteran Affairs, Washington, DC (US); The Regents Of The University Of California, Oakland, CA (US)

(72) Inventors: Lawrence Chan, San Francisco, CA (US); Tyson Kim, San Francisco, CA (US); William Rasper, Cleveland, OH (US)

(21) Appl. No.: 18/936,590

(22) Filed: Nov. 4, 2024

Related U.S. Application Data
(60) Provisional application No. 63/645,629, filed on May 10, 2024.
(60) Provisional application No. 63/596,010, filed on Nov. 3, 2023.

(51) Int. Cl. A61B 3/12; A61B 90/50

(52) U.S. Cl. CPC A61B 3/12; A61B 2090/502; A61B 2560/0425

(57) ABSTRACT

An ophthalmoscope for viewing an eye of a patient, including an optical device with a housing having a bottom window, a rear window, and a front wall including a reflective inner surface. A top wall may include a first portion, and a second portion wherein the first portion includes a reflective inner surface. A pair of side walls extends between side edges of the bottom window, the rear window, the top wall, and the front wall. The reflective inner surfaces of the top wall and the front wall are configured to direct light rays that enter the bottom window from the eye of the patient out of the rear window into the eyes of the ophthalmologist.

### CROSS-REFERENCE TO RELATED APPLICATIONS

This application claims priority to and the benefit of the filing date of U.S. Provisional Patent Application Nos. 63/645,629, filed May 10, 2024, and 63/596,010, filed Nov. 3, 2023, the entirety of each of which is hereby incorporated by reference herein.

### FIELD

This application is generally related to medical devices and procedures and, more particularly, to optical devices and methods of use thereof that facilitate the use of binocular indirect ophthalmoscopes in an ergonomic fashion during eye examination.

### BACKGROUND

Ophthalmoscopy is an examination performed by ophthalmologists in which the inside of the back of the eye, which is also known as the fundus or posterior segment, is observed. Examination of this portion of the eye is utilized to help detect issues such as, but not limited to, proliferative diabetic retinopathy in which abnormal blood vessels within the eye can grow over the retina, bleed into the vitreous, etc., leading to degraded or lost vision. A device often used by an ophthalmologist for such examinations is a binocular indirect ophthalmoscope (BIO) 10, as shown in FIGS. 9 and 10. A typical ophthalmoscope 10 includes a housing 18 that is adjustably received on a headband 12. A top portion 19 of the housing 18 includes a light source (not shown) disposed therein, and a bottom portion 26, or binocular portion, of the housing 18 includes a pair of eye pieces 22 on a back wall thereof and a viewing window 24 on a front wall thereof. As best seen in FIG. 10, the ophthalmoscope 10 is worn on the head 6 of the ophthalmologist 8 and includes a plurality of tension knobs 14 to adjust the fitting, a first angle knob 16 to adjust the angle of the housing 18, a second angle knob 20 to adjust the mirror (not shown) inside the housing 18, and an illumination knob 17 to adjust the intensity of the light source.

Referring specifically to FIG. 11, during a typical examination the patient 4 is either lying down or fully reclined. The ophthalmologist 8 positions a condensing lens 7 adjacent the patient's eye 2 in order to gather the light rays coming from the patient's eye 2 which are normally divergent due to the powers of the cornea. The condensing lens 7 directs the light rays upwardly toward the viewing window 24 of the ophthalmoscope 10 and, therefore, the eyes of the ophthalmologist. In order to receive the light rays in the viewing window 24, the ophthalmologist 8 must look downwardly toward the patient, thereby bending the neck downwardly for prolonged periods of time. The noted position of the ophthalmologist is known to cause repetitive use injuries within the profession.

From the foregoing, one recognizes a need amongst ophthalmologists for a device which allows for better posture, such as seated posture or erect standing posture while looking straight ahead, increased patient comfort, shorter and more efficient examinations and treatments of the patient, and a lower incidence of occupation-associated inquiries amongst the ophthalmologists.

### SUMMARY

Embodiments of the present disclosure provide an ophthalmoscope for use by an ophthalmologist when viewing an eye of a patient. The disclosed ophthalmoscopes allow the doctor to maintain an erect posture when performing an examination of the eyes of the patient. In one aspect, an embodiment of an ophthalmoscope in accordance with the disclosure may include an optical device with a housing having a bottom window, a rear window that extends upwardly from the bottom window, and a front wall that extends upwardly from the bottom window, wherein the front wall includes a reflective inner surface and forms an interior angle with the bottom window. A top wall may include a first portion that extends from the rear window at an interior angle, and a second portion that extends between the first portion of the top wall and the front wall, wherein the first portion of the top wall includes a reflective inner surface. A pair of side walls extends between corresponding side edges of the bottom window, the rear window, the top wall, and the front wall. Additionally, the reflective inner surfaces of the first portion of the top wall and the front wall are configured to direct light rays that enter the bottom window from the eye of the patient out of the rear window into the eyes of the ophthalmologist.

In some embodiments, the present disclosure relates to an ophthalmoscope for use by an ophthalmologist when examining the eye of a patient. The ophthalmoscope may include a binocular assembly having a housing with an entry window disposed on a front wall of the housing, and a pair of eyepieces disposed on a rear wall of the housing. The eyepieces are configured for placement adjacent the eyes of the ophthalmologist, and an optical device is adjacent the entry window of the housing so that light rays that enter the optical device from the eye of the patient are directed into the eyes of the ophthalmologist. In yet other embodiments, any configuration of reflective surfaces that directs the light rays from the patient's eye to the doctor's eyes may be used.

Additional advantages of the disclosed devices, systems, and methods will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIGS. 1A and 1B are perspective side views of a mounting clip and an optical device, respectively, of an ophthalmoscope in accordance with an embodiment of the present disclosure.

FIG. 2 is a perspective side view of the mounting clip and optical device shown in FIGS. 1A and 1B attached to the housing of an ophthalmoscope in accordance with the present disclosure.

FIG. 3 is a schematic side view of the ophthalmoscope shown in FIG. 2.

FIG. 4A is a side view of an exemplary mounting clip. FIG. 4B is a front view of the mounting clip of FIG. 19A.

FIG. 5A is a front view of an exemplary mounting clip. FIG. 5B is a side view of the mounting clip of FIG. 20A.

FIG. 6A is a side view of a housing of the optical device. FIG. 6B is a front view of the housing of FIG. 21A.

FIG. 7 is a side view of a portion of an ophthalmoscope with a mounting clip thereon for attaching the optical device.

FIG. 8A is a perspective view of an ophthalmoscope in accordance with an alternative version of the present disclosure. FIG. 8B is an exploded view of the ophthalmoscope of FIG. 8A.

FIG. 9 is a perspective view of a prior art ophthalmoscope.

FIG. 10 is a perspective depiction of the ophthalmoscope shown in FIG. 9 on the head of an ophthalmologist.

FIG. 11 is a depiction of a doctor inspecting the eye of a patient while using the prior art ophthalmoscope shown in FIGS. 9 and 10.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15 degrees, up to 10 degrees, up to 5 degrees, or up to one degree (above or below) of the particularly stated angular value can be included within the scope of those aspects.

The word “or” as used herein means any one member of a particular list and, unless context dictates otherwise, in alternative aspects, can also include any combination of members of that list.

In the following description and claims, wherever the word “comprise” or “include” is used, it is understood that the words “comprise” and “include” can optionally be replaced with the words “consists essentially of” or “consists of” to form another embodiment.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

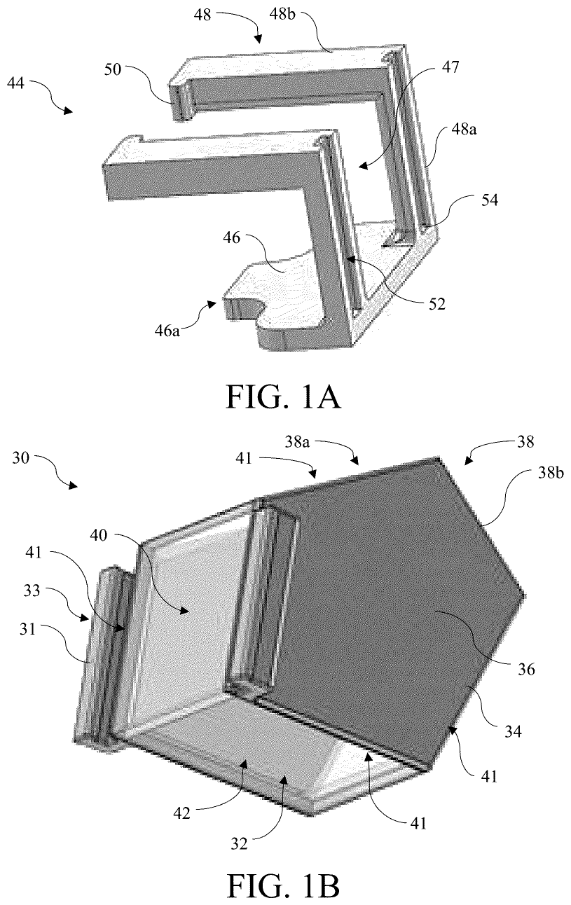

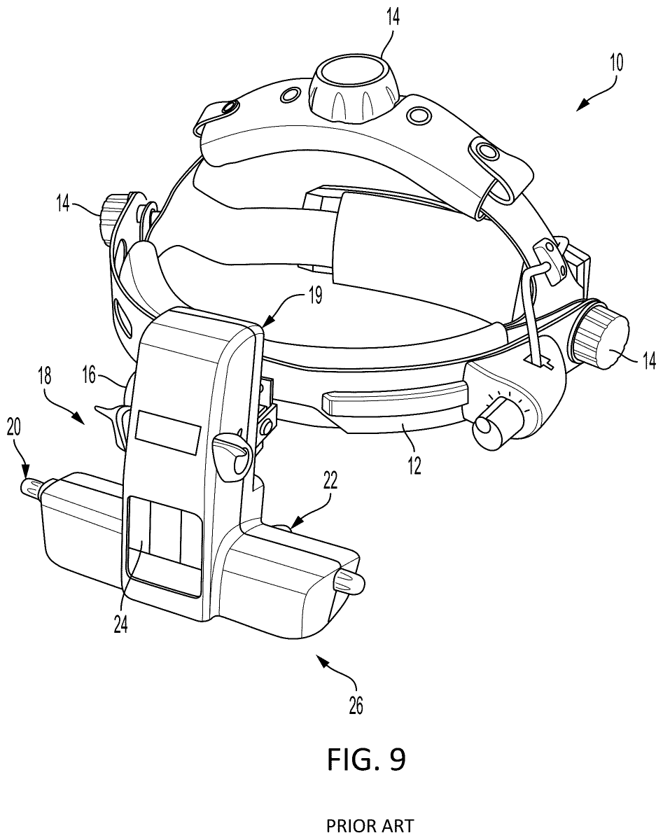

Referring now to the figures, an embodiment of an ergonomic binocular indirect ophthalmoscope in accordance with the present disclosure includes an optical device 30 and a mounting clip 44, as shown in FIGS. 1A and 1B, that are configured for use in conjunction with an ophthalmoscope 10, as shown in FIG. 9. The mounting clip 44 can permit modularity, adapting the optical device 30 for use with a particular ophthalmoscope having particular geometry. Referring specifically to FIG. 1B, the optical device 30 includes a bottom window 42 (or bottom wall), a front wall 34 extending upwardly from a front edge of the bottom window 42, a rear window 40 (or rear wall) extending upwardly from a rear edge of the bottom window 42, and a top wall 38 that extends between the top edge of the rear window 40 and the top edge of the front wall 34. As discussed in greater detail below, the top wall 38 can include a first portion 38a that is adjacent the top edge of the rear window 40, and a second portion 38b that is adjacent the top edge of the front wall 34, wherein the first portion 38a and the second portion 38b form an obtuse angle therebetween. A pair of parallel side walls 36 can each extend between corresponding edges 41 of the bottom window 42, the rear window 40, the top wall 38, and the front wall 34. As shown, a pair of elongated flanges 31 can each extend vertically along a corresponding side edge 41 of the rear window 40. As best seen in FIG. 1B, the bottom window 42 and rear window 40 may each be completely transparent, or in alternative embodiments, the rear window 40 and the bottom window 42 may comprise smaller portions of the entire areas of the bottom and rear walls of the housing 32.

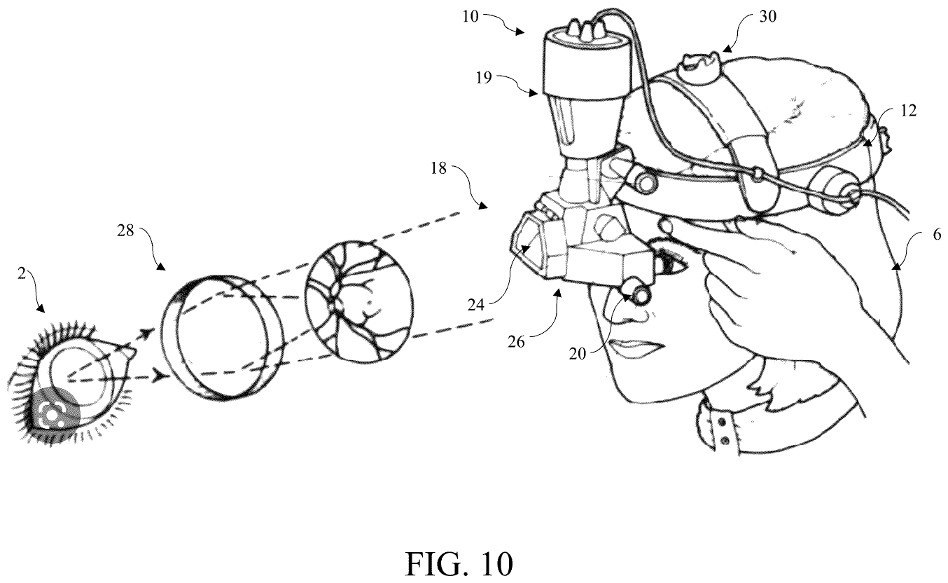

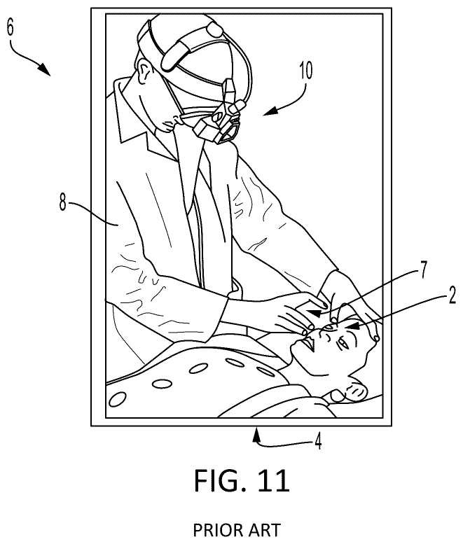

Referring specifically to FIG. 1A, the mounting clip 44 is provided for releasably securing the optical device 30 to a corresponding binocular indirect ophthalmoscope (BIO) 10 (FIGS. 9 and 10). In exemplary aspects, the mounting clip 44 optionally includes a base plate 46 and a pair of parallel or substantially parallel arms 48 extending upwardly from a top surface thereof. Each arm 48 can include a first portion 48a extending vertically from the base plate 46 and a second portion 48b that is parallel or substantially parallel to the base plate 46 and extends rearwardly from the distal end of the first portion 48a. As shown, a catch 50 extends inwardly from the distal end of each second portion 48b, although in alternative embodiments the catch 50 may extend downwardly from the distal end of the second portion 48b of each arm. The front surface of the first portion 48a of each mounting arm 48 can include an elongated groove 52 formed therein. A stop surface 54 can be formed at a bottom end of each elongated groove 52. Each elongated groove 52 can be configured to slidably receive the correspondingly (e.g., complementarily) shaped distal portion 33 of the corresponding flange 31 of the optical device 30 therein. For example, the distal portion 33 can have a dovetail fit within the elongated groove 52. As such, the optical device 30 can be removably secured to the mounting clip 44 (for example, using a double dovetail slide) using a tight and accurate fit. The stop surfaces 54 can be configured to prevent further downward motion of the optical device 30 with regard to the mounting clip 44 by abutting the bottom surfaces of the corresponding distal portions 33 of the flanges 31. Optionally, it is contemplated that the mounting clip 44 can be configured for complementary engagement with a plurality of different configurations or models of optical devices.

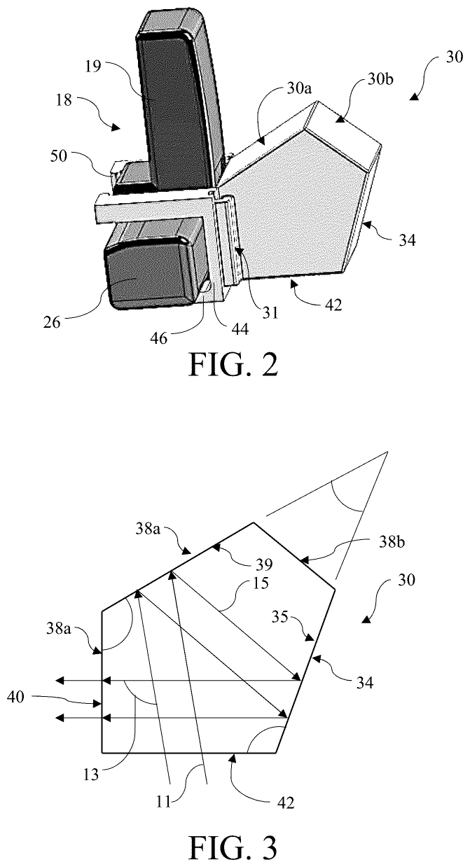

As shown in FIG. 9, a typical BIO 10 may include a housing 18 that is adjustably mounted to a headband 12, the headband 12 being configured to adjustably fit the ophthalmoscope 10 on the head 6 of an ophthalmologist 8, as shown in FIG. 10. Frequently, the housing 18 of an ophthalmoscope 10 includes a horizontally elongated binocular portion 26 and a more narrow and vertically oriented top portion 19 that is configured to house a light source therein. The width of the bottom binocular portion 26 is dictated by the fact that the rear of the housing 18 must be wide enough to accommodate two eyepieces 22 for viewing by the ophthalmologist. As shown in FIGS. 2 and 3, the mounting clip 44 of the disclosed embodiment can be configured to be removably secured to the housing 18 of the ophthalmoscope 10 by receiving the top portion 19 of the housing 18 between the mounting arms 48 so that the catch 50 of each mounting arm 48 extends inwardly beyond a rear surface of the housing 18 of the ophthalmoscope. The base plate 46 of the mounting clip 44 is urged downwardly with respect to the housing 18 until the rear edge 46a of the mounting clip extends downwardly beyond the bottom edge of the binocular portion 26 of the housing 18. At this point, the bottom edge 46a may move rearwardly until the top side of the base plate 46 is adjacent the bottom surface of the housing 18. The gap 47 formed between the vertical portions 48a of the mounting arms 48 can ensure that the vertical portions 48a are disposed on opposite sides of the viewing window 24 of the ophthalmoscope 10, thereby ensuring vision of the ophthalmologist is uninhibited.

With the mounting clip 44 in position, the optical device 10 can be slidably secured to the mounting clip 44 by positioning the bottom end of each distal portion 33 of each flange 31 thereof in the corresponding (e.g., complementary) elongated groove 52 of the mounting clip 44. The optical device 30 is urged downwardly so that the distal portion 33 of each flange 31 is slidably received in the corresponding elongated groove 52 until the bottom end of each flange 31 abuts the corresponding stop surface 54 disposed at the bottom end of the elongated groove 52. When the optical device 30 is fully seated within the mounting clip 44, the rear window 40 of the optical device 30 can be parallel or substantially parallel to and adjacent to the viewing window 24 of the ophthalmoscope 10, as shown in FIG. 10. In alternate embodiments, the dispositions of the flanges 31 and elongated grooves may be reversed, i.e., the flanges 31 being disposed on the mounting clip 44 and the elongated grooves 52 being formed in the housing of the optical device 30.

Referring now to FIGS. 2 and 3, the optical device 30 includes mirrored surfaces on the interior of the housing 32 that are configured to direct light rays that enter the bottom window 42, the incident light rays 11, out through the rear window 40 and into the viewing window 24 of the ophthalmoscope 10. Specifically, the first portion 38a of the top wall 38 includes mirrored surface 39 that is configured to direct incident light rays 11 that enter the housing 32 through the bottom window 42 toward a mirrored surface 35 that is disposed on the inner surface of the front wall 34. The reflected light rays 15 from the mirrored surface 35 of the front wall 34 are then directed toward the rear window 40 of the optical device 30 and into the viewing window 24 of the ophthalmoscope 10. As shown, in the present embodiment, the inclusive angle 13 between the incident light rays 11 and the reflected light rays 15 is an obtuse angle. This configuration of mirrored surfaces allows an ophthalmologist that is wearing an ophthalmoscope 10 that includes the disclosed optical device 30 to remain in an erect posture and look straight ahead into the binocular portion 26 of the ophthalmoscope 10, yet view the eye of the patient that is disposed directly beneath them. As such, the ophthalmologist is not required to crane his neck downwardly so that light rays from the patient's eye directly enter the viewing window 24 of the ophthalmoscope, as is shown in FIG. 10 and is required when using prior art ophthalmoscopes. Optionally, in exemplary aspects, and as described further below, the angle 13 can range from about 90 degrees to about 130 degrees, or from about 90 degrees to about 115 degrees.

Although various angles may be utilized between the walls and windows of the optical device 30, the presently disclosed embodiment can optionally be constructed such that the bottom window 42 and the rear window 40 form a 90 degree inclusive angle, whereas the front wall 34 and the bottom window 42 form an obtuse inclusive angle (e.g., of approximately 110 degrees), and first portion 38a of the top wall 38 and the rear window 40 also form an obtuse inclusive angle (e.g., of approximately 110 degrees). However, interior angles formed by the front wall 34 and the bottom window 42, and by the first portion 38a of the top wall 38 and the rear window 40, have been found to operate well in the range of 100 degrees to 120 degrees. Optionally, in exemplary aspects, it is contemplated that the inclusive angle formed between the front wall 34 and the bottom window 42 can be selectively adjusted within an angular range of about 90 degrees to about 130 degrees (for example, using at least one knob or other actuator that is coupled to the front wall and/or bottom window and configured to selectively rotate or pivot the front wall and/or bottom window). Additionally, or alternatively, in further optional aspects, it is contemplated that the inclusive angle formed between the first portion 38a of the front wall 38 and the rear window 40 can be selectively adjusted within an angular range of about 90 degrees to about 130 degrees (for example, using at least one knob or other actuator that is coupled to the first portion of the front wall and/or the rear window and configured to selectively rotate or pivot the first portion of the front wall and/or rear window). In use, it is contemplated that adjustment of the angular orientation of the mirrored surfaces as disclosed herein can provide a viewing angle ranging from about 90 degrees to about 130 degrees, optionally, about 90 degrees to about 115 degrees (measured relative to a center axis of the bottom window 42, which can optionally be a vertical axis when oriented directly over a subject). In addition to adjustment of the viewing angle by way of adjusting the interior mirrored surfaces of the optical device 30, the angle of the housing 18 of the ophthalmoscope 10 with respect to the ophthalmologist's head may be adjusted with the first angle knob 16. Moreover, further adjustment of the viewing angle is offered by adjusting the interior mirror (not shown) of the ophthalmoscope 10 with the second adjustment knob 20.

In exemplary aspects, as shown in FIGS. 1B-3, the optical device can optionally have a five-sided or generally five-sided cross-sectional profile. In some optional aspects, the optical device can comprise a penta-mirror configuration.

Further alternative embodiments of the present disclosure provide the ability to invert the image of the patient's ocular anatomy as viewed by the ophthalmologist through the ophthalmoscope 10. For example, the provision of an optical inverter within the optical device 30 may facilitate intraocular procedures such as, but not limited to, vitrectomy, subretinal and intravitreal injections, drug delivery, and the like. However, in some exemplary aspects, the image of the patient's ocular anatomy can be maintained in the same orientation as when viewed by the ophthalmologist when using a known ophthalmoscope. Further alternative embodiments of the present disclosure may provide varying degrees of magnification of the patient's ocular anatomy to facilitate various procedures, whereas even further embodiments may include wavelength filters that allow an ophthalmologist to highlight and observe various aspects of the patient's ocular anatomy, pathology, presence of drugs, etc.

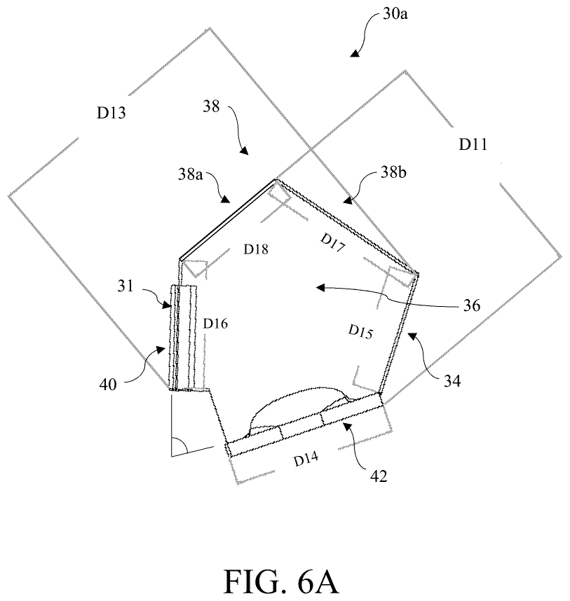

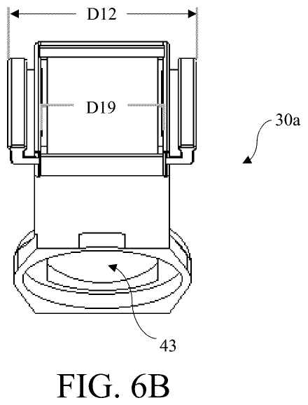

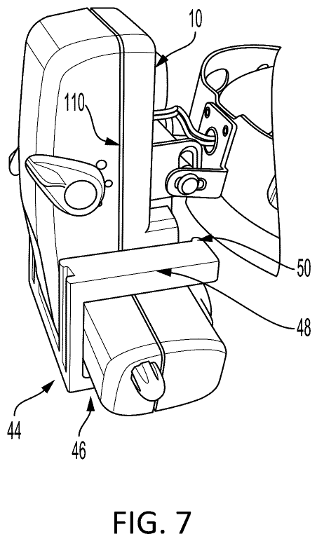

An alternative embodiment of an ergonomic BIO in accordance with the present disclosure includes an optical device 30a and a mounting clip 44a, as shown in FIGS. 6A and 6B. Referring specifically to FIGS. 6A and 6B, the optical device 30a includes a bottom window 42 (or bottom wall), a front wall 34 extending upwardly from the bottom window 42, a rear window 40 (or rear wall) extending upwardly from the bottom window 42, and a top wall 38 that extends between the rear window 40 and the front wall 34. In further aspects, the top wall 38 can include a first portion 38a that is adjacent the rear window 40, and a second portion 38b that is adjacent the front wall 34, wherein the first portion 38a and the second portion 38b form an obtuse angle therebetween. The optical device 30a of FIGS. 6A-6B differs from the previously discussed embodiment 30 (FIG. 1B-3) primarily in that the inclusive angle formed between the bottom wall 32 and the rear window 40 is an acute angle rather than a right angle, as best seen in FIG. 6A. As such, an additional connecting wall 41 can be provided to connect the bottom wall 32 to the rear window 40. Note, although the connecting wall 41 can optionally be excluded by extending both the bottom wall 32 and the rear wall to the point at which they intersect, this can undesirably increase the overall size of the housing of the optical device 30a. The dimensions may be modified as desired to allow for securing the mounting clip to various configurations and models of known ophthalmoscopes.

A pair of parallel side walls 36 can each extend between corresponding edges of the bottom window 42, the rear window 40, the top wall 38, and the front wall 34. As shown, a pair of elongated flanges 31 can each extend vertically along a corresponding side edge 41 of the rear window 40. The bottom window 42 and rear window 40 may each be completely transparent, or in alternative embodiments, the rear window 40 and the bottom window 42 may comprise smaller portions of the entire areas of the bottom and rear walls of the housing 32. For example, in the embodiment shown, a magnification lens 43 forms the transparent portion of the bottom wall 32.

Referring specifically to FIGS. 1A, 5A and 5B, a mounting clip 44a for securing the optical device 30a to an ophthalmoscope 10 (FIG. 10) can include a base plate 46 and a pair of parallel or substantially parallel arms 48 extending upwardly from a top surface thereof. Each arm 48 can include a first portion 48a extending vertically from the base plate 46 and a second portion 48b that is parallel or substantially parallel to the base plate 46 and extends rearwardly from the distal end of the first portion 48a. As shown, a catch 50 extends inwardly from the distal end of each second portion 48b, although in alternative embodiments the catch 50 may extend downwardly from the distal end of the second portion 48b of each arm. As shown, the front surface of the first portion 48a of each mounting arm 48 can include an elongated groove 52 formed therein. A stop surface 54 can be formed at a bottom end of each elongated groove 52. Each elongated groove 52 can be configured to slidably receive the correspondingly (e.g., complementarily) shaped distal portion 33 of the corresponding flange 31 of the optical device 30a therein. As such, the optical device 30 can be removably secured to the mounting clip 44a, as shown in FIGS. 5A and 5B. The stop surfaces 54 can be configured to prevent further downward motion of the optical device 30a with regard to the mounting clip 44a by abutting the bottom surfaces of the corresponding distal portions 33 of the flanges 31.

The mounting clip 44a is secured to the housing 18 of an ophthalmoscope 10 by receiving the top portion 19 of the housing 18 between the mounting arms 48 so that the catch 50 of each mounting arm 48 extends inwardly beyond a rear surface of the housing 18 of the ophthalmoscope. The base plate 46 of the mounting clip 44 is urged downwardly with respect to the housing 18 until the rear edge 46a of the mounting clip extends downwardly beyond the bottom edge of the binocular portion 26 of the housing 18. At this point, the bottom edge 46a may move rearwardly until the top side of the base plate 46 is adjacent the bottom surface of the housing 18. The gap 47 formed between the vertical portions 48a of the mounting arms can ensure that the vertical portions 48a are disposed on opposite sides of the viewing window 24 of the ophthalmoscope, thereby ensuring vision of the ophthalmologist is uninhibited. Note, the dimensions of the mounting clip 44a shown in the figures are merely representative of a clip for use with a given embodiment of an ophthalmoscope. The dimensions may be altered as desired to allow the disclosed optical devices 30/30a to be utilized with various configurations or models of known ophthalmoscopes. For example, it is contemplated that the same optical device 30/30a can be used with different types of ophthalmoscopes by modifying the dimensions of the clip (or by substituting one clip size for another clip size), without the need for any further changes to the optical device and the ophthalmoscope.

With the mounting clip 44a in position, the optical device 30a can be slidably secured to the mounting clip 44a by positioning the bottom end of each distal portion 33 of each flange 31 thereof in the corresponding (e.g., complementary) elongated groove 52 of the mounting clip 44a. The optical device 30a is urged downwardly so that the distal portion 33 of each flange is slidably received in the corresponding elongated groove 52 until the bottom end of each flange 31 abuts the corresponding stop surface 54 disposed at the bottom end of the elongated groove 52. When the optical device 30a is fully seated within the mounting clip 44, the rear window 40 of the optical device 30 can be parallel or substantially parallel to and adjacent to the viewing window 24 of the ophthalmoscope 10.

Although various angles may be utilized between the walls and windows of the optical device 30a, the presently disclosed embodiment can be constructed such that the bottom window 42 and the rear window 40 form an acute inclusive angle 13 between the incident light rays 11 and the reflected light rays 15 (FIG. 3), whereas the front wall 34 and the bottom window 42 form an obtuse inclusive angle (e.g., of approximately 110 degrees), and first portion 38a of the top wall 38 and the rear window 40 also form an obtuse inclusive angle (e.g., of approximately 110 degrees). However, interior angles formed by the front wall 34 and the bottom window 42, and by the first portion 38a of the top wall 38 and the rear window 40, have been found to operate well in the range of 100 degrees to 120 degrees.

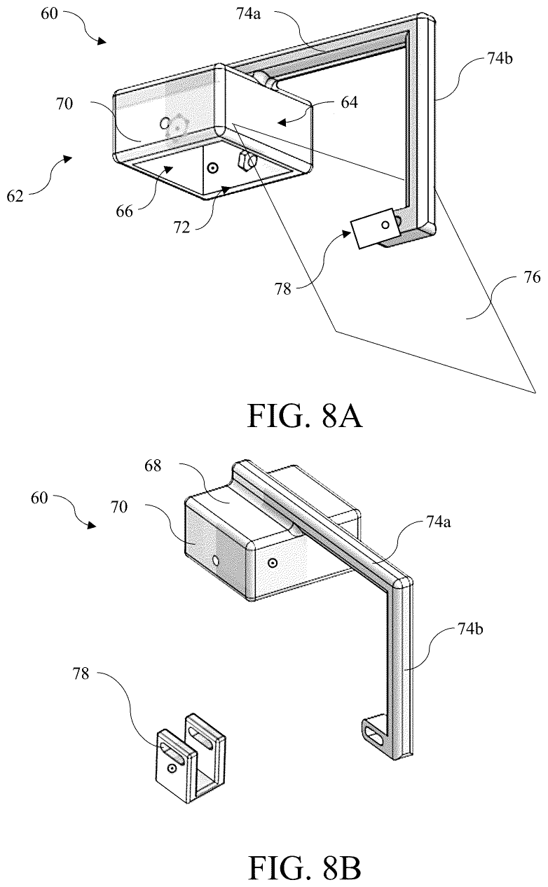

As shown in FIGS. 8A and 8B, yet another alternative embodiment of an ergonomic indirect ophthalmoscope in accordance with the present disclosure includes an optical device 60 including housing 62 that is configured to be received on a top portion of a housing 18 of an ophthalmoscope 10, an arm 74, and a mirror 76. As shown, the arm 74 can be configured to position a mirror 76 so that light rays from the eye of the patient are directed from a vertical path to a horizontal path that enters the viewing window 24 of the ophthalmoscope 10. As shown, the housing 62 of the optical device 60 can include a front wall 64, a rear wall 66 that is parallel or substantially parallel to the front wall 64, a pair of side walls 70 extending therebetween, and a top wall 68 that connects the top edges of the other walls. An aperture 72 can be formed in the bottom of the housing 62 and can be configured to slidably receive the top portion 19 of the housing 18 (FIG. 2) of the ophthalmoscope 10 therein. The arm 74 can include a first portion 74a that extends horizontally outward to the front of the housing 60, and a second portion 74b that extends vertically downward from a distal end of the first portion 74a. A mirror 76 can be connected to the bottom end of the second portion 74b of the arm 74 by a pivot assembly 78 that allows the angle of the mirror 76 with respect to the viewing window 24 of the ophthalmoscope 10 to be adjusted. In exemplary aspects, it is contemplated that the angle of the mirror 76 can be adjusted within a range from about 90 degrees to about 130 degrees to allow for comfortable viewing across a range of positions for a variety of anatomies. When the mirror is properly positioned, the ophthalmologist may maintain an erect posture and look straight ahead as the mirror 76 will reflect the vertical light rays from the eye of the patient into the viewing window 24 of the ophthalmoscope 10. In an alternative embodiment, it is contemplated that the rigid arm 74 may be replaced by a flexible arm 74 that retains the desired shape after being properly adjusted by the ophthalmologist.

Referring to FIG. 7, the mounting clip 44 can be attached to the ophthalmoscope 10 by sliding over the front of the device and along the sides so that the catch 50 engages a back face of the ophthalmoscope 10. Optionally, the back face of the ophthalmoscope can define or comprise a slot or other geometry that is configured to engage (e.g., complementarily engage) the catch(es) 50 of the clip 44. The catches 50 of the mounting clip 44 can keep the device from sliding forwardly off the ophthalmoscope 10. The geometries of the mounting clip 44 can be selected based on the dimensions of the ophthalmoscope 10 so the mounting clip does not slide laterally and also fits snugly in the vertical dimension. For example, the mounting clip 44 can comprise opposed arms 48 that bias against opposite sides of the ophthalmoscope 10. The arms 48 can be vertically spaced from the base plate 46 by a predetermined distance to inhibit vertical movement of the clip relative to the ophthalmoscope 10. Optionally, in further aspects, and with reference to FIG. 7, the ophthalmoscope 10 (e.g., the housing of the ophthalmoscope) can have a groove 110, and the catches 50 of the mounting clip 44 can engage said groove 110 to secure the clip. More generally, the mounting clip can be configured to securely attach to the ophthalmoscope 10.

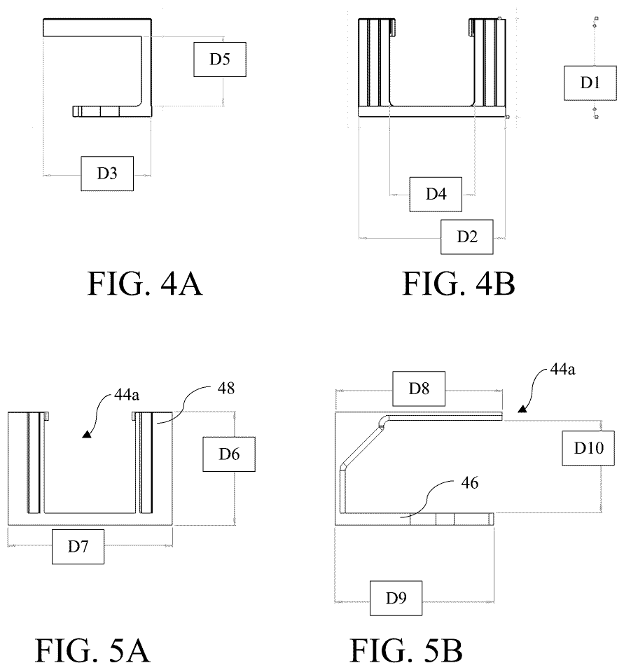

FIGS. 4A and 4B illustrate side and front views of an exemplary clip 44. In exemplary aspects, height D1 can be about 45 mm±20 mm, or 45 mm±4 mm, or about 47 mm. In exemplary aspects, width D2 can be about 70 mm±50 mm, or 70 mm±6 mm, or about 70 mm. In exemplary aspects, depth D3 can be about 35 mm±25 mm, or 35 mm±12 mm, or about 52 mm. More generally, the clip can have dimensions and geometry, including D4 and D5, selected based on the particular ophthalmoscope for which the clip is intended to be used.

In some aspects, the ophthalmoscope 10 can comprise a laser. In these aspects, clip 44 can be configured for use with the ophthalmoscope comprising the laser. FIGS. 5A and 5B illustrate front and side views, respectively, of such an exemplary clip. In exemplary aspects, height D6 can be about 56±20 mm, or 56 mm±4 mm, or about 56 mm. In exemplary aspects, width D7 can be about 81±50 mm, or 81 mm±6 mm, or about 81 mm. In exemplary aspects, depth D8 can be about 81 mm±25 mm, or 81 mm±12 mm, or about 82 mm. More generally, the clip can have dimensions and geometry, including D9 and D10, selected based on the particular ophthalmoscope for which the clip is intended to be used.

FIGS. 6A and 6B illustrate side and front views, respectively, of a housing for the optical device. In exemplary aspects, height D11 can be 95 mm±50 mm, or 95 mm±5 mm, or about 61 mm. In exemplary aspects, width D12 can be 60±25 mm, or 61±5 mm, or about 81 mm. Optionally, the width D12 can be at least 35 mm. In further aspects, the width D12 can be at least 40 mm. In exemplary aspects, depth D13 can be 35 mm±50 mm, or 90 mm±10 mm, or about 88 mm. In exemplary, optional aspects, window dimension D19 can be about 40 mm. In exemplary, optional aspects, dimension D14 can be about 60 mm. In exemplary, optional aspects, dimension D15 can be about 45 mm. In exemplary, optional aspects, dimension D16 can be about 46 mm. In exemplary, optional aspects, dimension D17 can be about 60 mm. In exemplary, optional aspects, dimension D18 can be about 45 mm.

In use, and as further disclosed herein, the mounting clip 44 can be attached to the BIO by sliding over the front of the device and securely attaching to the device (e.g., by hooking on to or otherwise engaging the back face or by engaging a groove defined by the device). The catches (e.g., hooks) of the clip can keep the device from sliding back off, while the geometries of the clip can match the dimensions of the BIO device so the clip does not slide laterally and also fits snugly in the vertical dimension. It is contemplated that the mounting clip can work in the same fashion regardless of whether a laser-based BIO or non-laser BIO is used—however, it is understood that the geometry of the clip can be modified to match the geometry and angle of the mirror of the laser BIO or non-laser BIO. Thus, it is contemplated that both versions of the clips can allow for the mirror housing to interface with the BIO with a tight, accurate fit every time, and the mirror housing can be easily transferred between and among any type or brand of BIO as long as the associated clip is provided.

The Laser BIO version works in the same way, but matches the geometries of the laser bio, thus the clip is longer and has a diagonal extrusion to match the angle of the mirror on the laser bio.

Although the foregoing invention has been described in some detail by way of illustration and example for purposes of clarity of understanding, certain changes and modifications may be practiced within the scope of the appended claims. For example, dependent upon the shape of the housing of the ophthalmoscope, the mounting clip for the optical device may take many different forms. Additionally, it is contemplated that the disclosed optical devices may be secured to a corresponding ophthalmoscope by an adhesive, a flexible strap, a hook-and-loop fastener, etc. It is further contemplated that the optical device may be practiced through the use of a prism that is positioned adjacent the viewing window of the ophthalmoscope.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An ophthalmoscope comprising:
an optical device including a housing, the housing comprising:
   a bottom window;
   a rear window that extends upwardly from the bottom window;
   a front wall that extends upwardly from the bottom window, wherein the front wall includes a reflective inner surface and forms an interior angle with the bottom window; and
   a top wall including a first portion that extends from the rear window at an interior angle, wherein the first portion of the top wall includes a reflective inner surface; and
   a pair of side walls, each side wall extending between corresponding side edges of the bottom window, the rear window, the top wall, and the front wall,
wherein the reflective inner surfaces of the first portion of the top wall and the front wall are configured to direct a light ray that enters the bottom window from the eye of a patient out through the rear window.

Aspect 2: The ophthalmoscope of aspect 1, wherein an interior angle defined between the bottom window and the rear window is an acute angle or a right angle.

Aspect 3: The ophthalmoscope of aspect 2, wherein an interior angle formed between the first portion of the top wall and the rear window is an obtuse angle, and an interior angle formed between the front wall and the bottom window is an obtuse angle.

Aspect 4: The ophthalmoscope of aspect 3, wherein the obtuse angle formed between the first portion of the top wall and the rear window is in a range of 95° to 115°, and the obtuse angle formed between the front wall and the bottom window is in a range of 95° to 115°.

Aspect 5: The ophthalmoscope of aspect 2, further comprising a binocular assembly including a housing, an entry window disposed on a front wall of the housing of the binocular assembly, and a pair of eyepieces disposed on a rear wall of the housing of the binocular assembly, wherein the rear window of the optical device is disposed adjacent the entry window of the binocular assembly.

Aspect 6: The ophthalmoscope of aspect 5, further comprising a mounting clip including a base plate and a pair of mounting arms extending upwardly therefrom, wherein the housing of the optical device is removably secured to the mounting arms of the mounting clip, and the mounting arms of the mounting clip are configured to removably secure the optical device to the housing of the binocular assembly.

Aspect 7: The ophthalmoscope of aspect 6, wherein:
each mounting arm includes a vertical portion extending upwardly from the base plate and defining an elongated groove therein, and a horizontal portion extending rearwardly from a distal end of the vertical portion, and wherein the housing of the optical device includes a pair of elongated rails, each elongated rail extending along a corresponding side edge of the rear window, and
each elongated rail of the housing of the optical device is slideably received in the elongated groove of a corresponding mounting arm.

Aspect 8: The ophthalmoscope of aspect 7, wherein the vertical portion of each mounting arm of the mounting clip is disposed adjacent a corresponding side edge of the entry window of the binocular assembly, and wherein the base plate is disposed adjacent a bottom surface of the housing of the binocular assembly.

Aspect 9: The ophthalmoscope of aspect 5, further comprising a head band, wherein the binocular assembly is operably secured to the head band.

Aspect 10: The ophthalmoscope of aspect 9, further comprising a light source disposed within the housing of the binocular assembly.

Aspect 11: The ophthalmoscope of aspect 1, wherein the top wall further comprises a second portion that extends between the first portion of the top wall and the front wall.

Aspect 12: The ophthalmoscope of aspect 1, wherein at least two of the front window, rear window, first portion of the top wall, and front wall are angularly adjustable relative to each other.

Aspect 13: The ophthalmoscope of aspect 1, further comprising a mounting clip including a base and a pair of mounting arms extending upwardly therefrom, wherein the housing of the optical device is configured to be removably secured to the mounting arms of the mounting clip, and wherein the mounting arms of the mounting clip are configured to removably secure the optical device to a housing of a binocular assembly.

Aspect 14: The ophthalmoscope of aspect 13, further comprising at least one catch configured to engage the housing of the binocular assembly.

Aspect 15: The ophthalmoscope of aspect 14, wherein the at least one pair of catches comprises a pair of catches, each catch extending inwardly from respective arms of pair of the mounting arms.

Aspect 16: An ophthalmoscope for use by an ophthalmologist when viewing an eye of a patient, comprising:
a binocular assembly, comprising:
   a housing;
   an entry window disposed on a front wall of the housing; and
   a pair of eyepieces disposed on a rear wall of the housing,
an optical device adjacent the entry window of the binocular assembly so that a light ray that enters the optical device from the eye of the patient is directed into one or both eyes of the ophthalmologist.

Aspect 17: The ophthalmoscope of aspect 16, the optical device including a top wall, a rear wall extending downwardly from the top wall, a front wall extending downwardly from the top wall, and a bottom wall extending from the rear wall to front wall, wherein the rear wall of the optical device is disposed adjacent the entry window of the housing.

Aspect 18: The ophthalmoscope of aspect 17, further comprising a head band, wherein the binocular assembly is operably secured to the head band.

Aspect 19: The ophthalmoscope of aspect 18, further comprising a light source disposed within the housing of the binocular assembly.

Aspect 20: The ophthalmoscope of aspect 17, wherein a portion of an inner surface of each of the top wall and the front wall is reflective.

What is claimed is:

1. An ophthalmoscope comprising:
an optical device including a housing, the housing comprising:
a bottom window;
a rear window that extends upwardly from the bottom window;
a front wall that extends upwardly from the bottom window, wherein the front wall includes a reflective inner surface and forms an interior angle with the bottom window; and
a top wall including a first portion that extends from the rear window at an interior angle, wherein the first portion of the top wall includes a reflective inner surface; and
a pair of side walls, each side wall extending between corresponding side edges of the bottom window, the rear window, the top wall, and the front wall,
wherein the reflective inner surfaces of the first portion of the top wall and the front wall are configured to direct a light ray that enters the bottom window from the eye of a patient out through the rear window.

2. The ophthalmoscope of claim 1, wherein an interior angle defined between the bottom window and the rear window is an acute angle or a right angle.

3. The ophthalmoscope of claim 2, wherein an interior angle formed between the first portion of the top wall and the rear window is an obtuse angle, and an interior angle formed between the front wall and the bottom window is an obtuse angle.

4. The ophthalmoscope of claim 3, wherein the obtuse angle formed between the first portion of the top wall and the rear window is in a range of 95° to 115°, and the obtuse angle formed between the front wall and the bottom window is in a range of 95° to 115°.

5. The ophthalmoscope of claim 2, further comprising a binocular assembly including a housing, an entry window disposed on a front wall of the housing of the binocular assembly, and a pair of eyepieces disposed on a rear wall of the housing of the binocular assembly, wherein the rear window of the optical device is disposed adjacent the entry window of the binocular assembly.

6. The ophthalmoscope of claim 5, further comprising a mounting clip including a base plate and a pair of mounting arms extending upwardly therefrom, wherein the housing of the optical device is removably secured to the mounting arms of the mounting clip, and the mounting arms of the mounting clip are configured to removably secure the optical device to the housing of the binocular assembly.

7. The ophthalmoscope of claim 6, wherein:
each mounting arm includes a vertical portion extending upwardly from the base plate and defining an elongated groove therein, and a horizontal portion extending rearwardly from a distal end of the vertical portion, and wherein the housing of the optical device includes a pair of elongated rails, each elongated rail extending along a corresponding side edge of the rear window, and
each elongated rail of the housing of the optical device is slideably received in the elongated groove of a corresponding mounting arm.

8. The ophthalmoscope of claim 7, wherein the vertical portion of each mounting arm of the mounting clip is disposed adjacent a corresponding side edge of the entry window of the binocular assembly, and wherein the base plate is disposed adjacent a bottom surface of the housing of the binocular assembly.

9. The ophthalmoscope of claim 5, further comprising a head band, wherein the binocular assembly is operably secured to the head band.

10. The ophthalmoscope of claim 9, further comprising a light source disposed within the housing of the binocular assembly.

11. The ophthalmoscope of claim 1, wherein the top wall further comprises a second portion that extends between the first portion of the top wall and the front wall.

12. The ophthalmoscope of claim 1, wherein at least two of the front window, rear window, first portion of the top wall, and front wall are angularly adjustable relative to each other.

13. The ophthalmoscope of claim 1, further comprising a mounting clip including a base and a pair of mounting arms extending upwardly therefrom, wherein the housing of the optical device is configured to be removably secured to the mounting arms of the mounting clip, and wherein the mounting arms of the mounting clip are configured to removably secure the optical device to a housing of a binocular assembly.

14. The ophthalmoscope of claim 13, further comprising at least one catch configured to engage the housing of the binocular assembly.

15. The ophthalmoscope of claim 14, wherein the at least one pair of catches comprises a pair of catches, each catch extending inwardly from respective arms of pair of the mounting arms.

16. An ophthalmoscope for use by an ophthalmologist when viewing an eye of a patient, comprising:
a binocular assembly, comprising:
a housing;
an entry window disposed on a front wall of the housing; and
a pair of eyepieces disposed on a rear wall of the housing,
an optical device adjacent the entry window of the binocular assembly so that a light ray that enters the optical device from the eye of the patient is directed into one or both eyes of the ophthalmologist.

17. The ophthalmoscope of claim 16, the optical device including a top wall, a rear wall extending downwardly from the top wall, a front wall extending downwardly from the top wall, and a bottom wall extending from the rear wall to front wall, wherein the rear wall of the optical device is disposed adjacent the entry window of the housing.

18. The ophthalmoscope of claim 17, further comprising a head band, wherein the binocular assembly is operably secured to the head band.

19. The ophthalmoscope of claim 18, further comprising a light source disposed within the housing of the binocular assembly.

20. The ophthalmoscope of claim 17, wherein a portion of an inner surface of each of the top wall and the front wall is reflective.
